(19) United States
(12) Patent Application Publication
(10) Pub. No.: US 2009/0148487 A1
(43) Pub. Date: Jun. 11, 2009

(54) MOLDABLE BIOMATERIAL FOR BONE REGENERATION

(75) Inventors: Michael Siedler, Munich (DE); Klaus Hellerbrand, Geltendorf (DE); Andreas Schuetz, Stockdorf (DE); Sabrina Tauer, Grafelfing (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: SCIL TECHNOLOGY GMBH, Martinsried (DE)

(21) Appl. No.: 12/097,154

(22) PCT Filed: Dec. 14, 2006
(86) PCT No.: PCT/EP06/12082

§ 371 Date: Sep. 11, 2008

(30) Foreign Application Priority Data
Dec. 14, 2005 (EP) 05027401.8

(51) Int. Cl. A61F 2/00; A61K 9/14; A61K 38/18

(52) U.S. Cl. 424/422; 424/489; 424/499; 514/12

(57) ABSTRACT

The present invention is directed to a moldable biomaterial comprising a particulate solid porous material and a biodegradable paste material. The paste material and the particulate solid porous material form a matrix usable for the replacement or augmentation of bone. In various embodiments the matrix has a high structural integrity, which does not immediately or shortly after implantation collapse into an amorphous non-porous mass, maintains its porosity after implantation, shows biphasic degradation after implantation and/or has a good resistance against being washed out when it is applied to a wet opened implant site. Active agents can be incorporated in the moldable biomaterial of the present invention, such as bone growth factors. Kits, implants, method of manufacturing as well as medicinal uses are also provided.

The present invention is directed to a moldable biomaterial comprising a particulate solid porous material and a biodegradable paste material.

The paste material and the particulate solid porous material form a matrix usable for the replacement or augmentation of bone. In various embodiments the matrix has a high structural integrity, which does not immediately or shortly after implantation collapse into an amorphous non-porous mass, maintains its porosity after implantation, shows biphasic degradation after implantation and/or has a good resistance against being washed out when it is applied to a wet opened implant site.

Active agents can be incorporated in the moldable biomaterial of the present invention, such as bone growth factors.

Kits, implants, method of manufacturing as well as medicinal uses are also provided.

### BACKGROUND TECHNOLOGY

### Background of the Invention

Spinal fusion or spinal arthrodesis (e.g. lumbar spinal fusion) is commonly performed as a “last resort” in patients with chronic low back pain caused by degenerative changes and instability of the spine. One proposed method for managing low back pain caused by rupture or degeneration of the disc, collapse of the disc and dysarthrosis of a degenerative vertebral joint is removal of the vertebral disk and replacement by a porous device, which allows for bone growth and fusion of adjacent vertebrae. Such fusion techniques include anterior lumbar interbody fusion (ALIF), posterior lumbar interbody fusion (PLIF), transforaminal lumber interbody fusion (TLIF) in addition to posterolateral fusion, in which the fusion device is placed more posterolateral instead of a replacement of the disc.

Autogeneous bone grafts, which are harvested from the iliac crest, are the gold standard materials used in spinal fusion procedures. However, the downside of using the ileum as a harvest site for autogenous bone has been the creation of additional problems for the patient. These include routine post-incisional pain, complex regional pain disorders due to possible neuroma formation, infection, post-operative local hematoma, injury to the sacro-iliac joint, injury of pelvic ligament, and pelvic soft tissue problems. Furthermore, the autogenous bone graft has limited availability and inconsistent bone quality. Therefore, the use of autograft is going to be replaced by using bone substitutes in combination with growth factors such as those of the TGF-beta/BMP family including BMP-2, BMP-7, and GDF-5.

These factors are used in combination with collagen, collagen and carboxymethylcellulose such as OP-1 (Osigraft® (OP-1 Implant)/OP-1 Putty), Infuse® (InductOs®), collagen hydroxyapatite composites, calcium phosphate cement (Bone source®), true bone ceramics, beta-TCP, beta-TCP/hydroxyapatite composites (TCP/HA 15:85, TCP/HA 40:60), beta-TCP polymer composite materials including PLA-DX-PEG copolymer gels, or hydrogels.

WO 94/15653 discloses formulations comprising tricalcium phosphate (TCP), TGF-beta and collagen. The TCP is disclosed as being a delivery vehicle for the TGF-beta.

EP1 150 726 describes an osteogenic sponge useful for the induction of new bone growth comprising of a resorbable sponge material, an osteogenic factor and a particulate mineral.

In PCT/EP2005/006204 the present inventors provide an in-situ hardening paste comprising a plasticizer, a water insoluble polymer and a water insoluble solid filler, and optionally a pore forming agent which hardens after contact with an aqueous liquid such as water, or body fluid.

However, the conventional fusion devices or biomaterials have several disadvantages. They are for example not compression resistant and need a non physiological high concentration of bone growth promoting substances as described for collagen based materials with the resulting risk of undesired side effects. Other compositions (e.g. cements) collapse into an amorphous non-porous mass immediately or shortly after implantation and do not maintain a physical integrity of a porous matrix. Biodegradable implant materials such as beta-tricalcium phosphate granules as described in WO03/043673 or HA nano-suspensions, tend to be washed out or disintegrate when applied to a wet opened field such as a high bleeding surrounding. Biomaterials such as hydroxyapatite are non- or partially biodegradable and remain in the body over a long time.

Another limitation of hardening materials is the short timeframe between the hardening process and the application as well as lack of porosity (see e.g. classical calcium phosphate cements (CPCs) such as Biobon® (α-BSM, US2005/0089579), Biocement D and H, Biofill®, Bonesource®), Calcibon®, Cementek®, Mimics Biopex® and Norian® SRS®; more is described in PCT/EP2005/006204 which is incorporated in its entirety by reference herein). Most of these available CPC formulations are composed of two components that react and harden when mixed. The powder components are mixed with an aqueous solution some including an accelerator or promoter immediately before application to form an injectable paste. These pasty compositions are difficult to be stored in a pasty consistency for more than a few hours up to one or several weeks without hardening, in most cases even not more than 20 minutes to about 60 minutes or up to about 15 minutes dependent on the temperature at which the self-setting reaction occurs. CPCs including those comprising demineralised bone matrix (DBM) such as described in US2005/0084542 use two inorganic components for setting up the cement reaction after addition of a physiological aqueous fluid for scaffold formation in-vivo.

A further disadvantage of premixed pasty formulations is the necessary aseptic manufacturing, since it's not possible to sterilize the final product by common terminal sterilization methods such as gamma sterilization. One reason therefore is the damage of the active agent. Therefore the manufacturing is elaborative and highly costly.

In summary, despite the existence of biomaterials such as ceramic materials like beta-TCP, hydroxyapatite or mixtures of both, bone cements, composite materials including polymer based materials, or collagen as described above, there remains a need for further improvement of biomaterials and methods for improved bone augmentation in indications including spinal fusion, craniomaxillofacial reconstruction, joint reconstruction and fracture repair. There is a need for an improved biocompatible and biodegradable composition, which provides in-vivo a porous scaffold for cells infiltration and migration to replace the biomaterial by bony structures while reducing the burden for the organism. Preferably, the composition shall be a biomaterial or device overcoming one or more of the above disadvantages of the prior art conventional fusion devices or biomaterials.

Another object is the provision of an improved biocompatible and biodegradable composition, which can be adjusted to the defect site and provides in-vivo a porous scaffold for cells infiltration and bone replacement.

Another object of the present invention is the provision of an in-situ hardening biomaterial suitable for implantation into a subject in the need of bone augmentation by a composition being able to form a macroporous scaffold after being placed into the defect which hardens in-vivo.

Another object of the present invention is the provision of an in-situ hardening biomaterial suitable for implantation into a subject in the need of bone augmentation by a composition being able to form a macroporous scaffold after being placed into the defect which hardens in-vivo, wherein the moldable biomaterial is not a calcium containing cement.

Another object of the present invention is the provision of an in-situ hardening biomaterial suitable for implantation into a subject in the need of bone augmentation by a composition being able to form a macroporous scaffold after being placed into the defect which hardens in-vivo with an improved porosity and/or mechanical strength.

Another object of the present invention is the provision of an in-situ hardening biomaterial suitable for implantation into a subject in the need of bone augmentation by a composition being able to form a macroporous scaffold after being placed into the defect which hardens in-vivo, which can be easily manufactured and exhibits improved storage stability.

Another object of the present invention is the provision of an improved biocompatible and biodegradable composition with a sustained release of the active agent.

Another object underlying the present invention is the provision of an improved biocompatible and biodegradable composition suitable as a delivery system allowing a lower dose of the active agent compared to conventional devices.

Another object underlying the present invention is the provision of an improved bone graft substitute material designed for bony fusion such as long bone fusions or vertebral fusion.

Another object underlying the present invention is the provision of a spinal implant, which comprises an osteogenic component to promote bony fusion between adjacent vertebrae. Another object of the present invention is the provision of an improved bone graft substitute material for bone augmentation including maxillofacial bone augmentation and periodontal regeneration.

### SUMMARY OF THE INVENTION

Surprisingly, the present inventors were able to provide a moldable biomaterial solving these objects and corresponding methods for the production of said biomaterial.

Therewith, the present inventors provide a moldable biomaterial comprising a particulate solid porous material with an average particle size of 100-4000 μm and a biodegradable paste material.

The paste material and the particulate solid porous material form a matrix particularly advantageous for the replacement or augmentation of bone. The matrix maintains its structural integrity for a period of at least about two to three days after implantation and maintains its porous structure after implantation into a physiological environment in which bone replacement is occurring. By “structural integrity” it is meant that the shape and size of the implanted matrix is substantially maintained. This is due to the two component system, in which the particulate solid porous material forms a structure of a high mechanical strength and the paste material which holds the particulate solid porous material together.

The structural integrity of the moldable biomaterial of the present invention is in contrast to other paste like compositions of the prior art such as ceramic or nano-cristalline hydroxyapatite suspension, the structure of which immediately or shortly after implant collapses into an amorphous non-porous mass. It is advantageous that the matrix of the moldable biomaterial of the present invention also maintains its porosity after implantation, which is important for the bone replacement or augmentation process.

The moldable biomaterial of the present invention is a two-component system showing biphasic degradation after implantation in-vivo, i.e. each component, particulate solid porous material and biodegradable paste material, causes a different degradation kinetic. Due to the biphasic degradation, the moldable biomaterial of the present invention maintains a porous structure for improved bone formation after implantation. In addition, the biphasic degradation enables an improved sustained release or delivery of active agents such as bone growth inducing agents.

Preferably, the moldable biomaterial of the present invention has a biphasic degradation profile of one of the components which ends up into a triphasic degradation profile of the two component system after implantation in-vivo. The triphasic degradation profile may be caused by the different degradation kinetic of particulate solid porous material, the polymer component of the biodegradable paste material and the ceramic component of the biodegradable paste material, respectively.

One advantage of the moldable biomaterial of the present invention is that it has a moldable coherent consistency, which can be easily adopted to the site of application and remains at the place of application. In contrast to other biodegradable implant materials such as beta-tricalcium phosphate granules or HA nano-suspensions, the implant of the present invention has a good resistance against being washed out when it is applied to a wet opened implant site, such as a high bleeding surrounding.

Another advantage of the present invention is that a negative influence of the organic solvent onto the active substance contained in the implant material can be omitted by separating the biodegradable paste material comprising the organic solvent and the active substance comprising particulate solid porous material such as beta-tricalcium phosphate granules.

A further advantage of the present invention is an increased porosity of the moldable biomaterial compared to conventional pasty compositions with a reduced polymer content and therefore reduced burden for the organism. In addition, the mechanical stability of the moldable biomaterial is increased compared to the conventional pasty compositions.

By providing a kit comprising the two isolated components of the moldable biomaterial of the present invention the mixing of the two components immediately before use is possible. By mixture of the active substance containing ceramic material with the organic solvent comprising biodegradable paste material shortly before using the moldable biomaterial of the present invention the shelf-life of the active agent, such as a bone growth inducing protein, can be further increased compared to a formulation containing an organic solvent and the active agent already under storage conditions.

Another advantage of a kit is that due to the separation of the two components the biodegradable paste material can be terminal sterilized for example by gamma sterilization. It is an aspect of the present invention that though the polymer content of the implant material is decreased compared to a polymer paste, such as the paste of PCT/EP2005/006204, the implant material surprisingly exhibits a hardness after 2 hours, which is 2.5 fold higher than the hardness of the polymer paste without the addition of the porous ceramic material.

Other effects or advantages of the present invention are described in the following.

The embodiments of the invention are:

(1) A moldable biomaterial comprising
a) a particulate solid porous material with an average particle size of 100-4000 μm and
b) a biodegradable paste material.
   (1a) Preferably, the particulate solid porous material forms the inner structure of the moldable biomaterial and is responsible for the mechanical strength, whereas the biodegradable paste material holds the particulate solid porous material together. The biodegradable paste material further improves the mechanical strength of the particulate solid porous material. Thanks to the present invention the former particulate solid porous material is incorporated into a single structured body with improved mechanical properties compared to the particulate solid porous material (e.g. the free flowing ceramic granules) and the biodegradable paste material.
   (1b) More preferable, the moldable biomaterial is a bone replacement material.
   (1c) Even more preferably, the moldable biomaterial is water-free.
   (1d) Most preferably, the biodegradable paste material comprises a non-collagen based polymer.
   (1e) In another embodiment, the biodegradable paste material comprises a synthetic polymer.
   (1f) In a further embodiment, the polymer content of the moldable biomaterial is less than 35 wt %, more preferably less than 25 wt %, less than 15 wt %, most preferably about 10 to 15 wt %.
   (1g) In a further embodiment, the content of the particulate solid porous material and the water insoluble solid filler of the moldable biomaterial is more than 50 wt %, more preferably more than 55 wt %, most preferably about 58 to 62 wt %.
   (1h) In another embodiment, the amount of the solid material within the moldable biomaterial is at least 55 wt %, preferably between 55 wt % and 80 wt %, between 55 wt % and 70 wt %, between 55 wt % and 65 wt %, between 58 wt % and 62 wt %. Preferably the solid material within the moldable biomaterial and/or the particulate solid porous material is selected from calcium sulfate, calcium phosphate and bovine derived bone graft substitute material.
   (2) The moldable biomaterial of embodiment 1, wherein
the biodegradable paste material is a paste comprising
   i. a plasticizer, which is a water soluble or water miscible biocompatible organic liquid;
   ii. a water insoluble polymer, which is soluble in the plasticizer and which is biocompatible, biodegradable, and/or bioresorbable; and
   iii. a water insoluble solid filler, which is insoluble in the plasticizer,
wherein the paste is preferably injectable.
   (2a) The moldable biomaterial of embodiment 1, wherein
a) the particulate solid porous material comprises granules, preferably ceramic granules, made of calcium phosphate or calcium sulfate, more preferably tricalcium phosphate, most preferably beta-tricalcium phosphate, with an average particle size of 100-4000 μm, 100-3000 μm, 100-2000 μm, 100-1500 μm, 500-4000 μm, 500-3000 μm, 500-2000 μm, 500-1500 μm, or 500-1000 μm and
b) the biodegradable paste material is a paste comprising
   i. a plasticizer, which is a water soluble or water miscible biocompatible organic liquid;
   ii. a water insoluble polymer, which is soluble in the plasticizer and which is biocompatible, biodegradable, and/or bioresorbable; and
   iii. a water insoluble solid filler, which is insoluble in the plasticizer,
wherein the paste in one aspect is preferably injectable.
   (2b) Optionally, the biodegradable paste material is injectable and stable in its package and capable of hardening in-situ to form a solid implant upon contact with the aqueous medium or body fluid.
   (2c) Said particulate solid porous material of the present invention is a biodegradable, bioresorbable and/or biocompatible, preferably macroporous and/or microporous biomaterial, which is osteoconductive and by addition of an active agent such as a bone growth promoting substance or combinations thereof has also osteoinductive properties. It might increase the mechanical stability of the moldable biomaterial and remains as a matrix for cell infiltration and subsequent bone replacement after degradation of the biodegradable paste material of b) such as the polymeric component.

Preferably, the solid porous material has interconnecting pores.

Preferably, said particulate solid porous material is an inorganic calcium compound or a silicium dioxide based material such as a bioglass. More preferably said particulate solid porous material is a calcium phosphate, most preferably a tricalcium phosphate, beta-tricalcium phosphate, alpha-tricalcium phosphate, apatite, calcium phosphate containing cement, tetra-calcium phosphate, biphasic tricalcium phosphate/hydroxyapatite material (TCP/HA) or a combination or mixture thereof, most preferably a beta-tricalcium phosphate.

Preferably said particulate solid porous material has a granular appearance, more preferably as free flowing granules. Preferably the average particle size of said particulate solid porous material and it's preferred embodiments are 100-4000 μm, 100-3000 μm, 100-2000 um, 100-1500 μm, 500-4000 μm, 500-3000 μm, 500-2000 μm, 500-1500 μm, or 500-1000 μm.

In addition, the particulate solid porous material is optionally a carrier for an active agent as for example described in embodiment 6 below. Preferably, the active agent is at least partially uniformly or equally distributed onto the particulate solid porous material. Most preferably, the particulate solid porous material is homogenously or evenly coated with the active agent such as morphogenetic proteins including but not limited to BMP-2, BMP-7 or GDF-5. The active agents including BMP-2, BMP-7 or GDF-5 may be employed in the active forms known in the art, including their mature proteins or biological active fragments or variants thereof (e.g. the mature human BMP-2 protein with an N-terminal Alanin extension).

(2d) Said water insoluble solid filler in said biodegradable paste material in one embodiment comprises
a) an inorganic compound, and/or
b) an organic compound.

The inorganic compound in this embodiment preferably is a calcium compound, magnesiumoxide, magnesium hydroxide, magnesium carbonate, silicium dioxide or a combination or mixture thereof, more preferably a calcium sulfate, calcium carbonate or calcium phosphate, most preferably tricalcium phosphate, beta-tricalcium phosphate, alpha-tricalcium phosphate, apatite, calcium phosphate containing cement, tetra-calcium phosphate, biphasic tricalcium phosphate/hydroxyapatite (TCPIHA) or a combination or mixture thereof.

Said organic compound comprises chitosan, collagen, calcium alginate, poly(2-hydroxyethyl methacrylate), hyaluronic acid or derivatives thereof, cellulose or derivatives thereof, or starch or derivatives thereof.

Combinations of one or more compounds mentioned in (2d) are also encompassed.

The biodegradable paste material optionally comprises at least one further calcium containing water insoluble solid filler, preferably selected from the group of calcium sulfate, calcium carbonate, calciumhydrogenphosphate or hydroxyapatite.

(2e) Said water insoluble polymer in the biodegradable paste material in one embodiment comprises poly(alpha-hydroxy acids), poly(ortho esters), poly(anhydrides), poly(aminoacids), polyglycolid acid (PGA), polylactic acid (PLLA), poly(D,L)-lactic acid (PDLLA), poly(lactic-co-glycolic acid) (PLGA), poly(lactic-co-glycolic acid) polyethylene glycol (PLGA-PEG) copolymers, poly(3-hydroxybutyricacid) (P(3-HB)), poly(3-hydroxy valeric acid) P(3-HV), poly(p-dioxanone) (PDS), poly(epsilon-caprolactone) (PCL), polyanhydride (PA) polyorthoester, polyglactine, or copolymers, terpolymers, blockcopolymers, combinations, mixtures thereof.

Preferably, the water insoluble polymer is PLGA, preferably a water insoluble polymer with a lactic acid/glycolic acid ratio of less than 75:25, preferably 50:50.

Also preferably, the water insoluble polymer is an end-capped polymer. End-capped polymers comprise modified, but not free carboxyl end groups, which leads to a change of polarity compared to non-end-capped polymers.

Preferably, the water insoluble polymer is a non-end-capped polymer or a polymer with a free carboxyl endgroup. Such polymers may better interact with a polar, preferably positively charged active agent than end-capped polymers. This then leads to the advantage of an further sustained release compared to an end-capped polymer.

In one aspect, the water insoluble polymer content of the biodegradable paste material is equal or smaller than 40 wt %.

In another embodiment, the density of the biodegradable paste material composition is equal to or greater than 1.21 g/ml, preferable between 1.3 g/ml and 1.5 g/ml.

(2f) Said plasticizer in said biodegradable paste material in one embodiment comprises polyethylene glycol (PEG) 400, PEG 200, PEG 300, PEG 600, 1,3-butandiole, castor oil, N-methyl-2-pyrrolidone, 2-pyrrolidone, C2 to C6 alkanols, propylene glycol, solketal, acetone, methyl acetate, ethyl acetate, ethyl lactate, methyl ethyl ketone, dimethylformamide, dimethyl sulfoxide, dimethyl sulfone, tetrahydrofuran, decylmethylsulfoxide, oleic acid, propylene carbonate, N,N-diethyl-m-toluamide; 1-dodecylazacycloheptan-2-one or mixtures thereof.

Preferably, the plasticizer in said biodegradable paste material comprises polyethylene glycol (PEG) 400.

Preferably, the plasticizer content of the biodegradable paste material is 40-95 wt %, more preferably 40-55 wt %.

(2g) In the biodegradable paste material the ratio (i.e. weight ratio) of the water insoluble solid filler and the water insoluble polymer is preferably between 1:1 and 5:1, more preferably between 1:1 and 3:1, even more preferably approximately 1.5:1 as in a mixture containing less than 50 wt %, preferably 30 to 36 wt % water insoluble solid filler and less than 40 wt %, preferably 20-25 wt % water insoluble polymer.
(3) The moldable biomaterial of any of embodiments 1 or 2, which has a moldable consistency, and which is preferably capable of hardening in-situ to form a solid implant, preferably a solid porous implant, upon contact with an aqueous medium or body fluid.
(3a) More preferably, the moldable biomaterial of any of above embodiments, whereas the solid implant has interconnecting pores.
(4) The moldable biomaterial of any of embodiments 1 to 3, wherein the components a) and b) are used in a ratio in order to form a coherent product, preferably in a ratio of 1:0.3 wt % to 1:2 wt %, preferably 1:1 wt % to 1:2 wt %, more preferably 1:1.3 wt % to 1:1.7 wt %, most preferably 1:1.4 wt % to 1:1.6 wt %.

In a preferred embodiment the structure of the moldable biomaterial is a two component system of a) and b). The particulate solid porous material improves the mechanical strength of the system after hardening in-vivo, whereas the biodegradable paste material provides a coherent semi-solid structure which holds the particulate solid porous material together before and during application. After application to the implantation site the semi-solid coherent material hardens and links together the solid porous particles by forming at least partially solid bridges between the particles in-vivo. Thus, by combining the two components a coherent moldable material is generated, which is a locally fixed or stationary biomaterial in contrast to granular materials such as beta-TCP. This coherent moldable material will be transferred into a coherent scaffold for cell infiltration and subsequent bone formation after in-situ hardening within an aqueous solution or body fluid. The moldable feature of the biomaterial facilitates filling of various devices or bone formation in various applications such as bone void filling, critical size defects, long bone defects and spinal fusion. Preferably, a ratio of a) and b) of 1:0.3 wt % to 1:2 wt %, preferably 1:1 wt % to 1:2 wt %, more preferably 1:1.3 wt % to 1:1.7 wt %, most preferably 1:1.4 wt % to 1:1.6 wt % is used. These ratio allows the ideal binding of the ceramic particles to a coherent system vice versa achieving a maximum porosity of the final implant material as used for an indication such as spinal fusion. The ratio of the paste material to the ceramic particles in the final moldable biomaterial modulates the total porosity of the biomaterial after in-situ hardening and avoids a collapse of the material to promote the regeneration process. Even after degradation of the polymeric component a porous scaffold of the particular solid porous material remains at the place of application, which will than be replaced by newly formed tissue such as bone or cartilage.

(5) The moldable biomaterial of any of embodiments 1 to 4, wherein the paste of component b) comprises a water-soluble degradation regulating agent.
(5a) Said water soluble degradation regulating agent in the moldable biomaterial comprises in one embodiment one or more of a
(a) swelling agent, preferably cellulose derivatives;
(b) surfactant, preferably block copolymers of ethylene oxide and propylene oxide such as Pluronics® or Tween® 80; or
(c) porogenic agent such as trehalose, mannitol, sucrose, sorbitol, physiological amino acids, e.g. glycine, glutamin, arginine, sodium citrate, sodium succinate and sodium phosphates, sodium chloride, polyvinylpyrrolidon (PVP), solid PEGs such as PEG 4000, PEG 10000, sodium hydrogen carbonate, calcium sulfate or chitosan; or
(d) gas or gas forming agent such as calcium carbonate or sodium hydrogencarbonate
   (5b) The water soluble degradation regulating agent content in the biodegradable paste material is less than 10 wt %, preferably less than 5 wt %, more preferably between 1-4 wt %, most preferably 1.5-3.5 wt %, most preferably 2-3.5 wt % based on the total weight of the paste of component b).
   (5c) The water soluble degradation regulating agent in the biodegradable paste material is preferably carboxymethylcellulose, more preferably carboxymethylcellulose of less than 10 wt %, preferably less than 5 wt %, more preferably between 1-4 wt %, even more preferably 1-3.5 wt %, most preferably 2-3.5 wt % based on the total weight of the biodegradable paste material of component b).
   (5d) The water soluble degradation regulating agent in the biodegradable paste material preferably has an average particle size of less than 1000 μm, preferably between 25 to 1000 μm, more preferably 50 to 500 μm, most preferably 100 to 300 μm, preferably with a viscosity of 1500-2500 mPa*s, preferably with a degree of substitution between 0.2 and 1,3, more preferably between 0.6 and 1, most preferably of about 0.7.
   (6) The moldable biomaterial of any of embodiments 1 to 5, further comprising
c) an active agent, preferably a therapeutically effective amount of an active agent, most preferably, the active agent is a tissue regenerating agent, a bone growth factor, a bone inducing agent or a cartilage inducing agent.
   (6a) The active agent in the moldable biomaterial is preferably coated onto the particulate solid porous material or entrapped within the particulate solid porous material.
   (6b) In another aspect, the active agent is coated onto the water insoluble solid filler or dissolved or suspended in the plasticizer, preferably homogenous coated onto a water insoluble solid filler of the biodegradable paste material.
   (6c) Preferably, the moldable biomaterial without or preferably with active agent has osteoinductive and/or osteoconductive, cartilage or periodontal ligament regenerating properties in-vivo.
   (7) The moldable biomaterial of any of embodiments 1 to 6, wherein the active agent is selected from the group consisting of hormones, cytokines, growth factors, preferably bone growth factors, antibiotics and small molecules.
   (7a) In one aspect, the active agent is parathyroid hormone (PTH) and/or PTH 1-34 peptide.
   (7b) In another aspect, the active agent is an osteoinductive or cartilage inductive protein.
   (7c) In another aspect, the active agent is a member of the TGF-beta family or a member of the BMP or GDF family, preferably selected from BMP-1, BMP-2, BMP-3, BMP-4, BMP-5, BMP-6, BMP-7, BMP-8, BMP-9, BMP-10, BMP-11, BMP-12, BMP-13, BMP-14, BMP-15 or BMP-16; GDF-1, GDF-2, GDF-3, GDF-4, GDF-5, GDF-6, GDF-7, GDF-8, GDF-9, GDF-10 or GDF-11. Combinations of two or more of these active agents are also encompassed in this aspect, where appropriate.
   (7d) In another aspect, the active agent is the cartilage regenerating cartilage derived-retinoic acid-sensitive protein (CD-RAP).
   (7e) Preferably, said active agent is selected from BMP-2, BMP-7, and GDF-5.
   (8) The moldable biomaterial of any of embodiments 1 to 7, which contains 5 μg-2 mg active agent per ml biomaterial, preferably 250 μg-2 mg per ml, most preferably 250 μg-1 mg per ml.
   (9) The moldable biomaterial of any of embodiments 1 to 8, which shows biphasic degradation in-situ.

An advantage of the present invention is that the polymer and the immobilized particulate solid porous material form a composite matrix, which is particularly advantageous for the replacement or augmentation of bone. After about two to three days, during which the physiological integrity of the matrix is maintained, the polymer degradation increases and over several weeks a matrix structure of the porous solid ceramic is maintained within an environment in which substitution of the biomaterial by newly formed bone occurs.

The term “biphasic degradation” means a two step degradation, the initial degradation of the polymer and a second degradation phase, where the particulate solid porous material will be resorbed for example by cells such as osteoclasts and be replaced by newly formed bone. The second degradation period might allow a further release of an active agent for acceleration of the remodelling process. This degradation profile yields to a release pattern that can be divided in different or continuous release phases. Such release phases can for example consist of an initial release, a further release upon degradation and/or diffusion out of the polymer and a final release upon breakdown of the polymeric component.

(10) The moldable biomaterial of any of embodiments 1 to 9, which maintains a physical integrity for a period of at least 2 to 3 days after hardening in-situ and/or which maintains a porous granular structure after degradation of the polymeric component.
(10a) The moldable biomaterial in a preferred embodiment comprises:
(a) beta-tricalcium phosphate
(b) i. PEG 400
   ii. PLGA
   iii. calcium phosphate selected from the group of calcium phosphate containing cement, calcium carbonate, hydroxyapatite, calcium hydrogenphosphate, beta-tricalcium phosphate and alpha-tricalcium phosphate or a mixture thereof; and
   iv. optionally a carboxymethylcellulose sodium salt.
      (10b) The moldable biomaterial in a further preferred embodiment comprises:
(a) beta-TCP granules with an average particle size of 500-1000 μm, preferably with a total porosity of 20 to 70%;
(b) i. PEG 400: 40 to 50 wt %, preferably 40 to 45%;
   ii. PLGA: 20 to 25 wt %, preferably 22 to 25%;
   iii. calcium phosphate selected from the group of calcium phosphate containing cement and beta-tricalcium phosphate: 25 to 40 wt %, preferably 30 to 35%; and
   iv. optionally carboxymethylcellulose sodium salt.

The optional carboxymethylcellulose sodium salt component in embodiments (10a) and (10b) may preferably be included in an amount of less than 10 wt %, preferably less than 5 wt %, more preferably between 1-4 wt %, most preferably 2-3.5 wt % based on the total weight of the paste of component b).

The total porosity according to the present invention means the macro- and/or microporosity of the synthetic biomaterial such as the beta-TCP. The porosity can be determined by methods such as mercury porosimetry and microCT well known to the expert in the field.

Preferably, the beta-TCP is a phase pure beta-TCP to avoid undesired side effects during the degradation of the biomaterial. Phase purity can be determined by methods such as high resolution X-ray diffractometry as described for example in Tadic and Epple, (2004), Biomaterials 25: 987-994.

(11) A kit comprising the isolated components a) and b) of the moldable biomaterial as set forth in any of embodiments 1 to 10 or the isolated components a), b) and c) of the moldable biomaterial as set forth in any of embodiments 6 to 10.

Thanks to the present invention, the separation of the two components a) and b), the separation of a), b) and c) or the separation of b) and c) increases the stability of the active agent over time, therefore increasing the regeneration potential of the moldable biomaterial. This improves long-term storage and therefore a cost effective provision of the final product. Furthermore, the stability of the paste material might be further prolonged by using one or more primary packaging components such as blisters, glass vials to avoid absorption or diffusion of water into the biodegradable paste material commonly used in pharmaceutical preparations and well known to the expert in the field. Another advantage of the separation of both components in comparison with a ready to use product (e.g. a one component product) is that the industrial manufacturing of the moldable biomaterial is significantly simplified (e.g. by terminal sterilization) and less costly compared to other industrial manufacturing processes such as an aseptic manufacturing process.

(11a) In a preferred embodiment the kit might also contain an apparatus for application such as a syringe, an applicator, an injector gun, an attachment device, a device, a spinal fusion device, a minimal invasive application device, a spatula, a crucible, or combination thereof.
(12) An implant comprising the components a) and b) of the moldable biomaterial as set forth in any of embodiments 1 to 10 or the components a), b) and c) of the moldable biomaterial as set forth in any of embodiments 6 to 10, preferably a hardened implant, which is obtained upon contacting with an aqueous solution.
(13) A method of manufacturing a moldable biomaterial comprising mixing a paste comprising
i. a plasticizer, which is a water soluble or water miscible biocompatible organic liquid
ii. a plasticizer, which is a water soluble or water miscible biocompatible organic liquid;
iii. a water insoluble solid filler, which is insoluble in the plasticizer;
with a particulate porous material with an average particle size of 100-4000 μm, preferably a particulate porous material as described in the above embodiments.
so that the mixture has a moldable consistency, which is capable of hardening in-situ to form a solid porous implant upon contact with the aqueous medium or body fluid.
   (14) The method of embodiment 13, wherein the biodegradable paste material is dried to reduce water impurities and/or is manufactured using water-free components (i), (ii) and/or (iii).

The advantage of this manufacturing step is a further increase in stability of the paste material and the respective moldable biomaterial for example to avoid premature hardening, chemical alteration or chain cleavage of the polymer of the moldable biomaterial.

(15) Use of the moldable biomaterial of any of embodiments 1 to 10, of the kit of embodiment 11 or of the implant of embodiment 12 for the manufacture of a pharmaceutical composition or a medical device to be used for indications such as spinal fusion, long bone defects, critical size defects, non-union, joint relocation preferably knee or hip relocation, fracture repair, cartilage repair, full-thickness or partial-thickness defects, maxillofacial reconstruction, sinus floor augmentation, periodontal repair, periodontitis, degenerative disc disease, spondylolisthesis, bone void filling.
(15a) Preferably, the pharmaceutical composition or the medical device are to be used for fusing adjacent vertebrae. In this embodiment the pharmaceutical composition or the medical device are preferably to be inserted between adjacent vertebrae, optionally within a spinal implant such as a spinal fusion cage or spacer.

Spinal implants used for spinal surgery are well known to the expert in the field and are available in different configurations ranging from cylindrical or conical cages (threaded cages), box shaped or rectangular cages (non-threaded cages), horizontal cylinders (e.g. BAK cage), vertical rings (e.g Harms cage), open boxes (e.g. Brantigan cage), to solid rectangular parallel piped spacers for example the LT-Cage Lumbar Tapered Fusion Device, INTER FIX™ and INTER FIX™ Threaded Fusion Devices, as well as bioresorbable cages such as the Telamon Peek™ and Telamon Hydrosorb™ with or without pedicle screws and fixation devices (Advances in spinal fusion, Molecular Science, Biomechanics and Clinical Management, Marcel Dekker, Inc New York 2004). Different fusion techniques are further described above and known to experts in the field.

Preferably, the moldable biomaterial of the above embodiments is filled into a spinal implant so that the material fills out the voids or hollow structures to avoid fibrous tissue formation instead of bone formation. Optionally, the filled implant can be dipped, soaked or moistered in an aqueous liquid, body fluid or sodium chloride solution shortly before application into the body or tissue leading to a porous scaffold optimal for the migration of cells and tissue regeneration.

Alternatively, the pharmaceutical composition or the medical device can also be used for posterolateral fusion at one or multiple levels with or without internal fixation. In this embodiment the pharmaceutical composition or the medical device are preferably to be inserted posterolateral to the vertebrae, optionally with or without an internal fixation.

(15b) This embodiment takes into consideration that the moldable biomaterial of any of embodiments 1 to 10, of the kit of embodiment 11 or of the implant of embodiment 12 may be used in a method of spinal fusion, treating long bone defects, treating critical size defects, treating fractures, treating non-union, treating degenerative disc disease, treating spondylolisthesis, treating bone voids or in a method of fusing adjacent vertebrae, comprising inserting between adjacent vertebrae the moldable biomaterial of any of embodiments 1 to 10, the kit components of embodiment 11 or of the implant of embodiment 12 within a spinal implant such as a spinal fusion cage or spacer.

This embodiment takes also into consideration that the moldable biomaterial of any of embodiments 1 to 10, of the kit of embodiment 11 or of the implant of embodiment 12 may be used in a method of bone and/or cartilage induction, comprising inserting the moldable biomaterial of any of embodiments 1 to 10, the kit components of embodiment 11 or of the implant of embodiment 12.

(16) A moldable biomaterial manufactured by the method of embodiment 13 or 14.
(17) A pharmaceutical composition comprising the moldable biomaterial of any of embodiments (1) to (10), the kit of embodiment 11 or the implant of embodiment 12.
(18) Use of the moldable biomaterial of any of embodiments (1) to (10), the kit of embodiment 11 or the implant of embodiment 12 for the preparation of a pharmaceutical composition to be used for bone augmentation.
(18a) In a preferred embodiment said bone augmentation follows traumatic, malignant or artificial defects or is a prerequisite for the subsequent setting of an implant.
(19) Use of the moldable biomaterial of any of embodiments (1) to (10), the kit of embodiment 11 or the implant of embodiment 12 for the preparation of a pharmaceutical composition for treating bone defects.
(19a) In a preferred embodiment said bone defects are long bone defects, critical size defects, non-unions, defects after joint relocation such as knee and hip relocation, defects in the maxillofacial area or bone defects following apicoectomy, extirpation of cysts or tumors, tooth extraction, calvarian defects, bony defects of the neurocranium or viscerocranium, osteoporosis or surgical removal of retained teeth.
(20) Use of the moldable biomaterial of any of embodiments (1) to (10), the kit of embodiment 11 or the implant of embodiment 12 for the preparation of a pharmaceutical composition for treating degenerative, traumatic disc disease, spinal fusion, vertebral body fracture, vertebroplasty and kyphoplasty.
(21) Use of the moldable biomaterial of any of embodiments (1) to (10), the kit of embodiment 11 or the implant of embodiment 12 for the preparation of a pharmaceutical composition for treating bone dehiscence.
(22) Use of the moldable biomaterial of any of embodiments (1) to (10), the kit of embodiment 11 or the implant of embodiment 12 for the preparation of a pharmaceutical composition to be used for sinus floor elevation or augmentation of the atrophied maxillary or mandibular ridge.
(23) Use of the moldable biomaterial of any of embodiments (1) to (10), the kit of embodiment 11 or the implant of embodiment 12 for the preparation of a pharmaceutical composition for filling cavities, regeneration in periodontology and/or support guided tissue regeneration in periodontology.
(24) Use of the moldable biomaterial of any of embodiments (1) to (10), the kit of embodiment 11 or the implant of embodiment 12 for the preparation of a pharmaceutical composition for promoting chondrogenesis.
(25) Use of the moldable biomaterial of any of embodiments (1) to (10), the kit of embodiment 11 or the implant of embodiment 12 for the preparation of a pharmaceutical composition to be used for the treatment of at least one cartilage disease.

Preferably said bone disease is selected from the following diseases in which chondrogenic differentiation is involved: osteoarthritis, rheumatoid arthritis, injury of articular cartilage due to trauma, osteochondral defects, full-thickness or partial-thickness defects, maintenance of chondrocyte phenotypes in autologous chondrocyte transplantation, reconstruction of cartilage in the ear, trachea or nose, osteochondritis dissecans, regeneration of intervertebral disk or meniscus, bone fracture and/or osteogenesis from cartilage.

### DETAILED DESCRIPTION OF THE PRESENT INVENTION

The present invention is now described in detail by reference to the following definitions and to the description of the figures of the present invention.

### Definition of the Important Technical Terms

For the purpose of promoting an understanding of the principles of the invention, references will be made to certain embodiments thereof and the specific language used to describe the same. It will nevertheless be understood that no limitation of the scope of the invention is thereby intended, such alterations, further applications and modifications of the principle of the invention as illustrated herein being contemplated as would normally occur to one skilled in the art to which the invention relates.

### A the Moldable Biomaterial

The term “moldable biomaterial” means a biomaterial which can easily be adopted to any shape and form such as to fill hallow voids or cavities in a defect site or an implant. It includes a suspension, dispersion or liquid composition which preferably can be applied by a minimal invasive application or injection. It also includes a ductile paste-like material. Preferably, the moldable biomaterial is capable of hardening in a moist environment, preferably within the human body or in contact with human body fluids, i.e. is capable of hardening in-situ. The moldable biomaterial of the present invention distinguishes from other conventional biomaterials such as CPCs in being moldable prior to application of an aqueous solution such as saline solution or body fluids. In contrast to conventional self-hardening or self-setting reaction compositions such as cement compositions or poorly crystalline apatitic (PCA) calcium phosphate implant materials the moldable biomaterial of the present invention preferably comprises a particulate solid porous material with scaffolding properties instead of one or more reactive components for a chemical cement setting reaction. Preferably, the moldable biomaterial does not contain bone demineralized bone matrix (DBM) preferably in combination with calcium phosphate.

The term “water-free” means that the moldable biomaterial contains less than 5 wt %, more preferably less than 3 wt %, even more preferably less than 2 wt %, most preferably less than 1 wt % water determined by methods such as the Karl Fisher method. Preferably, the term water-free means that only trace amounts of free-water (e.g unbound water) exists in the moldable biomaterial. The reduced amounts of free-water may decrease the degradation rate of the polymer such as for example the PLGA, thus increasing the shelf-life of the moldable biomaterial.

Trace amounts of water means the amount of water which cannot be further reduced by standard manufacturing methods known to the expert in the field such as drying individual components, drying under reduced pressure or elevated temperature, known methods including thermal pretreatment of ingredients, vacuum drying, lyophillisation, and if appropriate by molecular sieve as well as using a packaging system with desiccants for packaging moisture sensitive pharmaceutical preparations.

The term “granular” such as granular material means discrete solid particles of a biomaterial such as sand, grains or powder with a size limit of at least 1 μm, preferably at least 50 μm, most preferably at least 100 μm.

The term “coherent” means sticking together or adhering. It is also encompassed that at least some for example particulate particles of the particulate porous material form bridges via the biodegradable paste material to at least some of their particulate neighbors to hold the particulate solid porous material together.

The term “in-situ hardening” as used in the present invention refers to forming a solid matrix after contact with an aqueous medium such as water, a physiological solution or body fluid after dissipation or dissolution of the organic solvent into the surrounding ex vivo as well as in an organism such as a human or an animal body or tissue. Dependent on the indication and use of the moldable biomaterial such a solid matrix would also encompass a matrix, preferably an implant, which matrix at least has a higher mechanical strength after getting into contact with a surrounding body fluid than the paste before application.

### B the Particulate Solid Porous Material

The term “particulate solid porous material” means a biodegradable, bioresorbable and/or biocompatible, preferably macroporous and/or microporous biomaterial, which is osteoconductive. It also means fine particles of a solid material such as calcium phosphate. A detailed description is further encompassed in the above embodiments.

### C The Biodegradable Paste Material

As indicated above, the present invention generally provides a biodegradable paste material including at least three components: a plasticizer, which is a water soluble or water miscible biocompatible organic liquid, a water insoluble polymer, which is biocompatible, biodegradable, and/or bioresorbable and soluble in the plasticizer, and a water insoluble solid filler, which is insoluble in the plasticizer, wherein the paste, is preferably injectable and stable in its package and hardens after being placed into the defect.

Preferably, stability in the package of the premixed biodegradable paste material is at least for several weeks, more preferably several months, most preferably at least one year. Stability can be understood as a consistency and moldability of the respective premixture without dramatic alterations in the consistency over time. The package comprise a commonly used waterproof package such as commonly used for parenteral applications in pharmaceutical applications.

The term “paste” as used in accordance to the present invention refers to a soft, smooth, thick mixture or material, or paste like entity administrable preferably using a syringe or minimal invasive application (i.e., capable of passing through a 16- to 18-gauge syringe), which comprises at least three components, preferably at least four components, as set forth in this specification. Preferably, the biodegradable paste material should be compatible with the active agent to avoid unwanted degradation and/or inactivation of the active agent. In at least some embodiments, the paste is a suspension, dispersion or liquid.

In a preferred embodiment the biodegradable paste material as well as the moldable biomaterial of the present invention is free of toxic substances. Preferably such toxic substances are already avoided in the production process, as their production requires additional expenditure due to required removal steps during the production process and necessary expensive means for highly sensitive chemical analysis.

The term “toxic substances”, in particular, encompasses those toxic organic solvents and additives which are used by the methods described in the art, which are classified by the ICH as class 2 solvents (ICH Topic Q 3 C Impurities: Residual Solvents) e.g. methylene chloride. Moreover, the international guidance for the development of therapeutic proteins requires that in the manufacturing process harmful and toxic substances should be avoided (for details see: International Conference on Harmonization (ICH), Topic Q3C; www. emea.eu.int/). However, the paste of the present invention is, advantageously, free of said class 1 classified toxic substances. Moreover the present invention contains only solvents classified as class 3 by the ICH Topic Q 3C and, therefore, therapeutically well acceptable and fulfills the requirements of the regulatory authorities.

Moreover, in a further preferred embodiment the biodegradable paste material or the moldable biomaterial of the invention is free of infectious material.

Preferably the same requirements as for solvents in common are valid for the plasticizer, the water insoluble solid filler and/or the water-soluble degradation regulating agent of the biodegradable paste as well as for the biodegradable paste itself and the moldable biomaterial of the present invention.

The variation of the concentration of the components of the biodegradable paste as well as of the moldable biomaterial of the present invention leads to an adaptation to a specific medical application by changes within the consistency of the paste or moldable biomaterial, hardening time in-situ, porosity and the mechanical properties of the final implant. Additionally the variation of these parameters is a potent means in adapting the release kinetic of the active agent by changed degradation behavior of the water insoluble polymer.

### D The Plasticizer

The term “plasticizer” according to the present invention means a water soluble or water miscible organic liquid or solvent which is pharmaceutically acceptable or a mixture thereof. Functions of the plasticizer are to dissolve the water insoluble biodegradable, biocompatible and/or bioresorbable polymer, to suspend the water insoluble solid filler material; or to dissolute the insoluble polymer additionally suspending the water insoluble solid filler. These functions may depend on the nature of the active agent.

Preferably, a function of the plasticizer is to reduce the glass transition temperature of the water insoluble biodegradable, biocompatible and/or bioresorbable polymer below a temperature where the biomaterial becomes moldable, more preferably, the glass transition temperature of the water insoluble biodegradable, biocompatible and/or bioresorbable polymer is below ambient temperature.

During the preferred in-situ hardening in contact with aqueous medium or body fluid the plasticizer diffuses out of the paste, leaving pores and leading to a form stable composite device or in-situ implant. Thereby the glass transition temperature of the polymer increases and the polymer solidifies and transfers the biomaterial into a mechanically stable implant. In a preferred embodiment the plasticizer is a water soluble or water miscible solvent. It can be a liquid; preferably the plasticizer is a water soluble polymer. Preferably the plasticizer has a low impact on the glass transition temperature of the water insoluble polymer in the in-situ hardened implant and is compatible with the active agent. Dependent on the water insoluble polymer a plasticizer selected from a group of plasticizers further defined below should be used with the lowest impact on the glass transition temperature of the polymer after setting.

The term “dissolving” means the dissolution or suspension of a substance in a liquid, yields to a homogenous distribution of the substance within the liquid.

Preferably said plasticizer is biocompatible. More preferably, said plasticizer is selected from the group consisting of polyethylene glycol (PEG) 400, PEG 200, PEG 300, PEG 600, 1,3 butandiole, castor oil, C2 to C6 alkanols, propylene glycol, solketal, acetone, methyl acetate, ethyl acetate, ethyl lactate, methyl ethyl ketone, dimethyl formamide, dimethyl sulfoxide, dimethyl sulfone, tetrahydrofuran, decylmethyl sulfoxide, oleic acid, propylene carbonate, N,N-diethyl-m-toluamide, 1-dodecylazacycloheptan-2-one or mixtures thereof.

Preferably the biodegradable paste of the present invention contains less than 60% of the plasticizer, more preferably less than 55%, even more preferably less than 50%, most preferably between 40% and 45%.

The term “biocompatible” means the ability of a material to perform with an appropriate host response in a specific application. Furthermore the term “biocompatible” means, that the material does not exhibit any toxic properties and that it does not induce any immunological or inflammatory reactions after application.

The term “biodegradable” specifies materials for example polymers, which break down due to macromolecular degradation with dispersion in-vivo but for which no proof exists for the elimination from the body. The decrease in mass of the biodegradable material within the body is the result of a passive process, which is catalyzed by the physicochemical conditions (e.g. humidity, pH value) within the host tissue.

The term “bioresorbable” specifies materials such as polymeric materials, which undergo degradation and further resorption in-vivo; i.e. polymers, which are eliminated through natural pathways either because of simple filtration of degradation by-products or after their metabolization. Bioresorption is thus a concept, which reflects total elimination of the initial foreign material. In a preferred embodiment said bioresorbable polymer is a polymer that undergoes a chain cleavage due to macromolecular degradation in an aqueous environment. The term “resorption” describes an active process.

### E The Water Insoluble Polymer

The term “water insoluble polymer” means a polymer not soluble in water, i.e. does not form a homogeneous phase when admixed with water, which is soluble in the plasticizer and capable of solidifying in aqueous media to form a solid implant in which the water insoluble solid filler is incorporated upon removal of the plasticizer into the surrounding tissue. Preferably said water insoluble polymer is a “biocompatible”, a “biodegradable” and/or a “bioresorbable” polymer. More preferably said water insoluble polymer is an aliphatic polymer preferably with a glass transition temperature above 30° C. of the pure polymer substance. The inherent viscosity (viscosity measured at 25° C., 0.1% in chloroform) of the polymers of the invention will range from about 0.1 dl/g to 5 dl/g, preferably from about 0.1 dl/g to 1 dl/g.

In another embodiment the polymer is a synthetic polymer.

Alternatively, said water insoluble polymer is selected from the group consisting of polyethylene (PE), polypropylene (PP), polyethylenerephthalate (PET), polyglactine, polyamide (PA), polymethylmethacrylate (PMMA), polyhydroxymethylmethacrylate (PHEMA), polyvinylchloride (PVC), polyvinylalcohole (PVA), polytetrafluorethylene (PTFE), polyetheretherketone (PEEK), polysulfon (PSU), polyurethane, polysiloxane or mixtures thereof.

More preferably, said polymer is selected from the group consisting of poly(alpha-hydroxy acids), poly (ortho esters), poly(anhydrides), poly(aminoacids), polyglycolid (PGA), polylactid (PLLA), poly(D,L-lactide) (PDLLA), poly(D,L-lactide-co-glycolide) or poly(L-lactide-co-glycolide) (PLGA), poly(lactic-co-glycolic acid) polyethylene glycol (PLGA-PEG) copolymers, poly(3-hydroxybutyricacid) (P(3-HB)), poly(3-hydroxy valeric acid) (P(3-HV)), poly(p-dioxanone) (PDS), poly(epsilon-caprolactone) (PCL), polyanhydride (PA), copolymers, terpolymers, blockcopolymers, combinations, mixtures thereof.

In another embodiment of the present invention the water insoluble polymer is an end-capped polymer. The term “end-capped polymer” means that the free carboxylic acid group of the linear polymer chain has been esterified with alcohols.

In another embodiment of the present invention the water insoluble polymer is a PLGA-PEG copolymer, preferably a PLGA-PEG diblock- or triblock-copolymer.

### F The Water Insoluble Solid Filler

The term “water insoluble solid filler” means a compound insoluble in water as well as in the plasticizer i.e. does not form a homogeneous phase when admixed with water or the plasticizer.

The water insoluble solid filler serves as matrix in the biodegradable paste material once the moldable biomaterial is hardened. Furthermore, the water insoluble solid filler can further increase the biocompatibility (e.g., cell attachment) to stabilize the local pH during degradation of the polymer.

Preferably said water insoluble solid filler is an inorganic or organic compound.

The term “calcium phosphate” encompasses compositions comprising calcium ions (Ca²⁺), phosphate ions (PO₃³⁻), optionally, further ions like hydroxyl ions (OH⁻), carbonate (CO₃²⁻) or magnesium (Mg²⁺) or other ions which are suitable for the water insoluble solid filler of the present invention.

### G The Water Soluble Degradation Regulating Agent

The term “water soluble degradation regulating agent” means a compound which is pharmaceutical acceptable and swellable or soluble in aqueous fluid such as water or body fluid which when added to the biodegradable paste material might increase the porosity of the moldable biomaterial ex vivo and in the organism. The porosity of the solid implant formed can for example be increased dependent on the amount of the water soluble degradation regulating agent used. Preferably, the water soluble degradation regulating agent increases the number of pores preferably macropores of a size sufficient for ingrowth of living cells into the in situ hardened material. More preferably, the water soluble degradation regulating agent allows an adjustment of the degradation of the polymeric component of the biodegradable paste material.

Another aspect of the degradation regulation agent can be the immobilization and/or enrichment of endogenous growth factors at the defect site further promoting the regeneration process such as but not limited to bone augmentation. The degradation regulation agent (e.g. swelling agents) can furthermore form a hydrogel within the moldable biomaterial when brought into contact with water, which resembles the properties of a natural occurring blood clot.

Water soluble degradation regulating agents of the present invention include e.g. sodium alginate, amylase, amylopectine, starch, hyaluronic acid, sodium hyaluronate, gelatine, collagen, carboxymethylcellulose, methylcellulose, carboxymethylcellulose calcium salt, carboxymethylcellulose calcium salt, hydroxylpropyl methylcellulose, hydroxybutylmethylcellulose, hydroxyethylcellulose, hydroxyethylcellulose, or methylhydroxyethylcellulose and derivatives thereof.

In another embodiment water soluble degradation regulating agents are surfactants, preferably block copolymers of ethylene oxide/sorbitan and propylene oxide such as Pluronics® or Tween® 80 (e.g., Polysorbate 80; Montanox® 80; Polyoxyethylene sorbitan monooleate).

More preferably the water soluble degradation regulating agents is a carboxymethylcellulose salt, most preferably a carboxymethylcellulose sodium salt, optimally with a particle size less than 1000 μm, more preferably with a particle size 25 to 1000 μm. Preferably the weight percentage of the carboxymethylcellulose sodium salt is less than 10 wt %, preferably less than 5 wt %, more preferably between 1-4 wt %, most preferably 2-3.5 wt % based on the total weight of the biomaterial paste component.

The term “particle size” according to the present invention means an average distribution of the size diameter of the material such as tricalcium phosphate or carboxymethylcellulose in microns (μm), which can be determined by sieving analysis or laser diffraction. A specific particle size range of material can for example be achieved by sieving

### H The Active Agent

The term “active agent” comprises a polypeptide or a small molecule drug.

It is to be understood that the active agents are preferably not aggregated and partially or entirely inactivated due to precipitation or micro-precipitation after implantation. This might be for example achieved by homogeneously coating on the particulate solid porous material as described in WO03/043673.

The term “homogeneously coated” or “homogeneously distributed” means that the active agent is homogeneously distributed on the inner and/or outer surface the particulate solid porous material.

Homogenous distribution is advantageous for efficient release and activity of the active agent into the tissue surrounding at the site of implantation. Moreover, it is to be understood that the active agent is not aggregated and partially or entirely inactivated due to precipitation or micro-precipitation, rather attachment of biologically active, non-aggregated proteins is to be achieved by homogenous coating.

The term “osteoconductive” refers to substrates that provide a favorable porous scaffolding for vascular ingress cellular infiltration and attachment, cartilage formation, and calcified tissue deposition. Osteoconductive materials may support osseous generation via the scaffolding effect.

The term “osteoinductive” refers to the capability of the transformation of mesenchymal stem cells into osteoblasts and chondrocytes. A prerequisite for osteoinduction is a signal, which is distributed by the moldable biomaterial into the surrounding tissues where the aforementioned osteoblast precursors become activated. Osteoinduction as used herein encompasses the differentiation of mesenchymal cells into the bone precursor cells, the osteblasts. Moreover, osteoinduction also comprises the differentiation of said osteoblasts into osteocytes, the mature cells of the bone. Moreover, also encompassed by osteoinduction is the differentiation of mesenchymal cells into chondrocytes. In particular in the long bones, the chondroblasts and the chondrocytes residing in the perichondrium of the bone can also differentiate into osteocytes. Thus, osteoinduction requires differentiation of undifferentiated or less-differentiated cells into osteocytes, which are capable of forming the bone. Thus, a prerequisite for osteoinduction is a signal, which is distributed by the moldable biomaterial into the surrounding tissues where the aforementioned osteocyte precursors usually reside.

The term “osteogenic” describes the synthesis of new bone by osteoblasts. In accordance with the present invention, preexisting bone cells or progenitor cells at the site of implantation or within the surrounding of the moldable biomaterial grow into the hardened moldable biomaterial using the structure of the hardened moldable biomaterial, especially formed during the hardening process, as a matrix onto which cells (e.g., bone cells) can adhere.

The proteins and peptides encompassed in the moldable biomaterial of the present invention preferably have osteoinductive properties in-vivo. For example, it is well known in the art that the Transforming Growth Factor-β (TGF-β) superfamily encompasses members, which have osteoinductive properties. Individual members of said TGF-β superfamily are listed infra. In conclusion, the osteoinductive proteins or peptides of the moldable biomaterial of the present invention after having been released from the carrier serve as an osteoinductive signal for the bone precursor cells of the tissue surrounding the site of implantation of the moldable biomaterial.

The term “osteoinductive polypeptide” refers to polypeptides, such as the members of the Transforming Growth Factor-beta (TGF-beta) superfamily, which have osteoinductive properties.

In a further preferred embodiment of the moldable biomaterial of the invention said osteoinductive protein is a member of the TGF-beta family.

The TGF-beta family of growth and differentiation factors has been shown to be involved in numerous biological processes comprising bone formation. All members of said family are secreted polypeptides comprising a characteristic domain structure. On the very N-terminus, the TGF-beta family members comprise a signal peptide or secretion leader. This sequence is followed at the C-terminus by the prodomain and by the sequence of the mature polypeptide. The sequence of the mature polypeptide comprises seven conserved cysteins, six of which are required for the formation of intramolecular disulfide bonds whereas one is required for dimerization of two polypeptides. The biologically active TGF-beta family member is a dimer, preferably composed of two mature polypeptides. The TGF-beta family members are usually secreted as proteins comprising in addition to the mature sequence the prodomain. The prodomains are extracellularly cleaved off and are not part of the signaling molecule.

In the context of the present invention, the term “TGF-beta family member” or the proteins of said family referred to below encompass all biologically active variants of the said proteins or members and all variants as well as their inactive precursors. Thus, proteins comprising merely the mature sequence as well as proteins comprising the mature protein and the prodomain or the mature protein, the prodomain and the leader sequence are within the scope of the invention as well as biologically active fragments or variants thereof. Whether a fragment of a TGF-beta member has the biological activity can be easily determined by biological assays described in the prior art.

More preferably, said member of the TGF-beta superfamily is a member of the BMP or GDF subfamily.

The osteoinductive polypeptide of the present invention is preferably selected from the group consisting of BMP-1, BMP-2, BMP-3, BMP-4, BMP-5, BMP-6, BMP-7, BMP-8, BMP-9, BMP-10, BMP-11, BMP-12, BMP-13, BMP-14, BMP-15, BMP-16, GDF-1, GDF-2, GDF-3, GDF-4, GDF-5, GDF-6, GDF-7, GDF-8, GDF-9, GDF-10 and GDF-11. Most preferably, the osteoinductive polypeptide is selected from the group consisting of BMP-2, BMP-7 and GDF-5.

Publications disclosing osteoinductive polypeptides include: OP-1 and OP-2: U.S. Pat. No. 5,011,691, U.S. Pat. No. 5,266,683, Ozkaynak et al. (1990) EMBO J. 9: 2085-2093; OP-3: WO94/10203 (PCT US93/10520); BMP2, BMP3, BMP4: WO88/00205, Wozney et al. (1988) Science 242:1528-1534); BMP5 and BMP6: Celeste et al. (1991) PNAS 87: 9843-9847; Vgr-1: Lyons et al. (1989) PNAS 86: 4554-4558; DPP: Padgett et al. (1987) Nature 325: 81-84; Vg-1: Weeks (1987) Cell 51: 861-867; BMP-9: WO95/33830 (PCT/US95/07084); BMP10: WO94/26893 (PCT/US94/05290); BMP-11: WO94/26892 (PCT/US94/05288); BMP12: WO95/16035 (PCT/US94/14030); BMP-13: WO95/16035 (PCT/US94/14030); GDF-1: WO92/00382 (PCT/US91/04096) and Lee et al. (1991) PNAS 88: 4250-4254; GDF-8: WO94/21681 (PCT/US94/03019); GDF-9: WO94/15966 (PCT/US94/00685); GDF-10: WO95/10539 (PCT/US94/11440); GDF-11: WO96/01845 (PCT/US95/08543); BMP-15: WO96/36710 (PCT/US96/06540); MP121: WO96/01316 (PCT/EP95/02552); GDF-5 (CDMP-1, MP52): WO94/15949 (PCT/US94/00657) and WO96/14335 (PCT/US94/12814) and WO93/16099 (PCT/EP93/00350); GDF-6 (CDMP-2, BMP13): WO95/01801 (PCT/US94/07762) and WO96/14335 and WO95/10635 (PCT/US94/14030); GDF-7 (CDMP-3, BMP-12): WO95/10802 (PCT/US94/07799) and WO95/10635 (PCT/US94/14030).

Preferably, active agents of the BMP or GDF subfamily, e.g. BMP-2, BMP-7, or GDF-5 refer to the preproform, to the proform or to the mature (e.g. BMP-2-BMP-7-, or GDF-5-) peptide, respectively. Moreover also encompassed are fragments and variants of said proteins having essentially the same biological activity, preferably osteoinductive properties.

Also encompassed within the present invention are variants of said proteins e.g. BMP-2 variants having essentially the same biological activity, which contain for example the mature BMP-2 protein sequence including N-terminal extensions such as an Alanin extension at the N-terminus as described by Ruppert et al. (1996), Eur. J. Biochem. 237: 295-302 and truncated forms of above mentioned polypeptides.

Preferably, the active agent is an unglycosylated protein, more preferably an E. coli derived recombinant protein. The advantage of unglycosylated protein is for example a prolonged immobilization at the defect site and/or a reduction of the required amount of the active agent such as rhBMP-2.

Also encompassed by the present invention are embodiments, wherein said active agent is selected from hormones, cytokines, growth factors, antibiotics and other natural and/or synthesized drug substances like steroids, prostaglandines etc.

Preferably, said active agent is parathyroid hormone (PTH) and/or PTH 1-34 peptide.

In another embodiment of the invention, the active agent is a “cartilage inductive” or “cartilage regenerating” protein. Preferred cartilage inductive proteins are MIA/CD-RAP (MIA, melanoma inhibitory activity, cartilage derived-retinoic acid-sensitive protein, EP 0710248, EP 1146897), OTOR (fibrocyte derived protein, FDP, MIA-like, MIAL) and TANGO 130 (Bosserhoff et al. (2004), Gene Expr. Patterns. 4: 473-479; Bosserhoff and Buettner (2003), Biomaterials 24: 3229-3234; Bosserhoff et al. (1997), Dev. Dyn. 208: 516-525; WO00/12762), more preferably human MIA/CD-RAP.

### I Implant

The term “implant” means a medical device, orthopaedic device, or biomaterial. Preferably, the implant is a spinal implant, implant for fracture repair, an implant for long bone defects, critical size defects and non-union, an implant for cartilage repair, maxillofacial reconstruction, joint reconstruction, implant for periodontal defects, an implant used as a bone void filler or an implant for other orthopedic surgical uses such as cages, plates, screws, pins, fixation devices.

The term “spinal implant” is further described above.

### DESCRIPTION OF THE FIGURES

Detailed aspects of the present invention are described in the following by reference to FIGS. 1-5.

FIG. 1 shows the inner and outer porosity of the two component moldable biomaterial of the present invention after in-situ hardening in an aqueous surrounding. In the image shown the composition was as follows: beta-tricalcium phosphate granules (40.0 wt %), polymer paste (60.0 wt %) comprising poly(-lactic-co-glycolic-acid) with a lactic-/glycolic acid ratio of 50:50 and a molecular weight of 13.6 kDa (22.2 wt %), polyethylene glycol 400 (44.4 wt %), beta-tricalium phosphate powder (33.3%).

Image A shows the outer surface of the two component moldable biomaterial after in-situ-hardening, exhibiting pores, which base exceptionally on voids between beta-tricalcium phosphate granules.

Image B shows the inner part of the material, exhibiting pores with a diameter larger than 100 μm, which is a basic requirement for integration of the implant material within the surrounding tissue.

The advantage of the two component moldable biomaterial of the present invention is that it has a moldable coherent consistency, which can be easily adapted to the site of application and remains at the place of application. In contrast to other biodegradable implant materials such as beta-tricalcium phosphate granules or HA nano-suspensions, the implant of the present invention has a good resistance against being washed out when it is applied to a wet opened field e.g. a surgical field, such as a high bleeding surrounding. In addition, the implant material can easily be used to be filled into implants such as various spinal fusion cages present on the market without leakage of the material and washing out effect. Furthermore the material has the properties of a coherent scaffold after implantation, which withstands the mechanical stress of the surrounding tissue. Another advantage over other injectable biomaterials is the porous structure of the implant material after in-situ hardening within the body or tissue and its compression resistance compared to biomaterials such as collagen based implants.

Larger monolithic biomaterials which form a porous matrix have the disadvantage that they cannot be applied in combination with hollow implants such as spinal fusion cages due their stiffness (bottleneck). Due to the moldable consistency and convenient application, the implant of the present invention can advantageously be used as a bone graft substitute biomaterial for filling of spinal implants such as a spinal fusion cage of various shapes, which forms a monolithic structure after in-situ hardening of the implant material within the cage.

FIG. 2 shows the additional outer porosity of the two component moldable biomaterial of the present invention after in-situ hardening, which additional outer porosity is achieved by swelling due to the addition of carboxymethylcellulose within the pasty component. The composition used had the following composition: beta-tricalcium phosphate granules (40.0 wt %), polymer paste (60.0 wt %) comprising of poly (-lactic-co-glycolic-acid) with a lactic-/glycolic acid ratio of 50:50 and a molecular weight of 13.6 kDa (21.6 wt %), polyethylene glycol 400 (43.1 wt %), beta-tricalium phosphate powder (32.3%) and carboxymethylcellulose sodium salt (3.0 wt %).

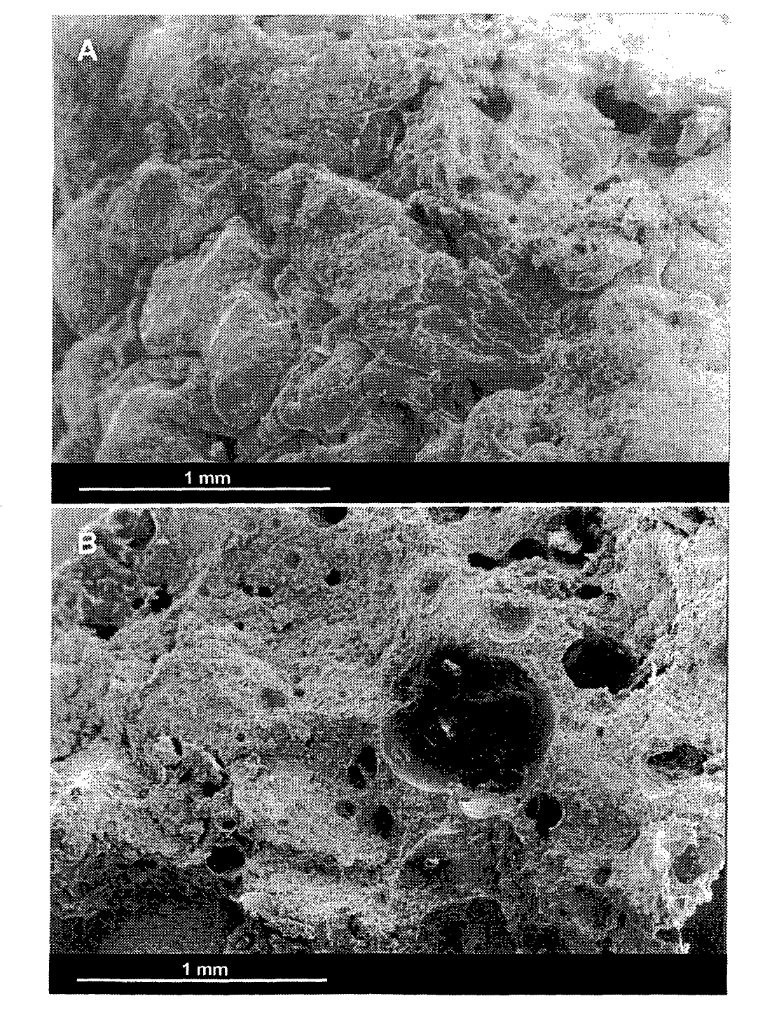

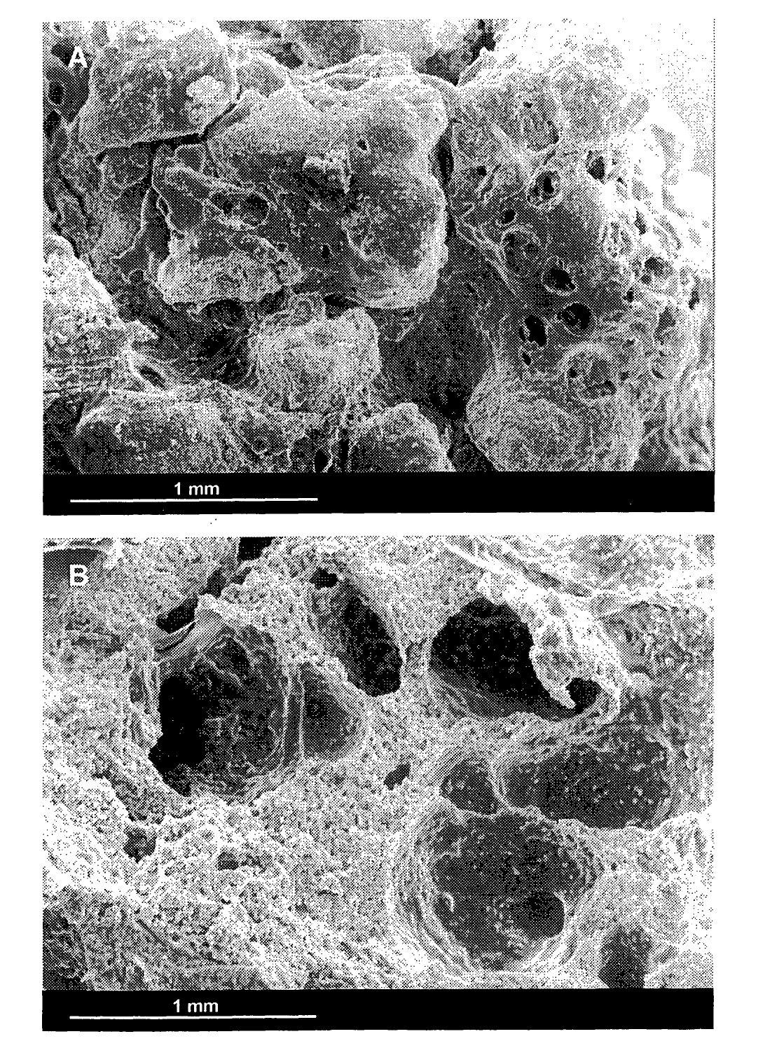

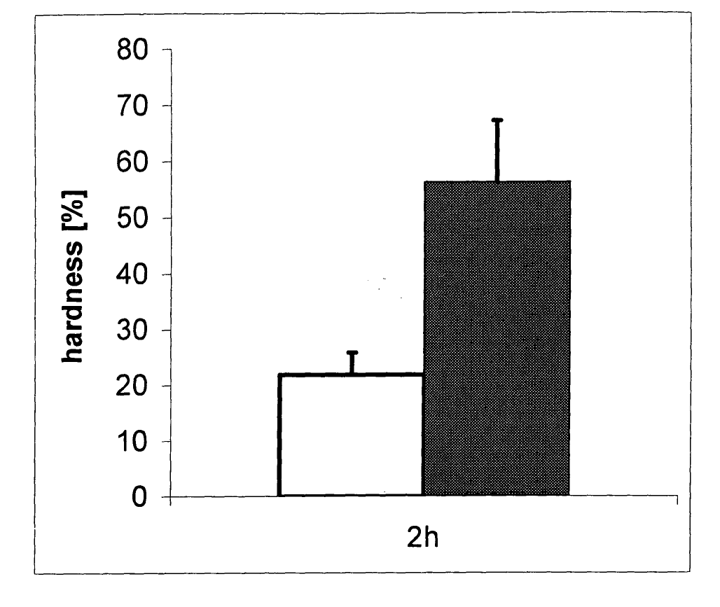

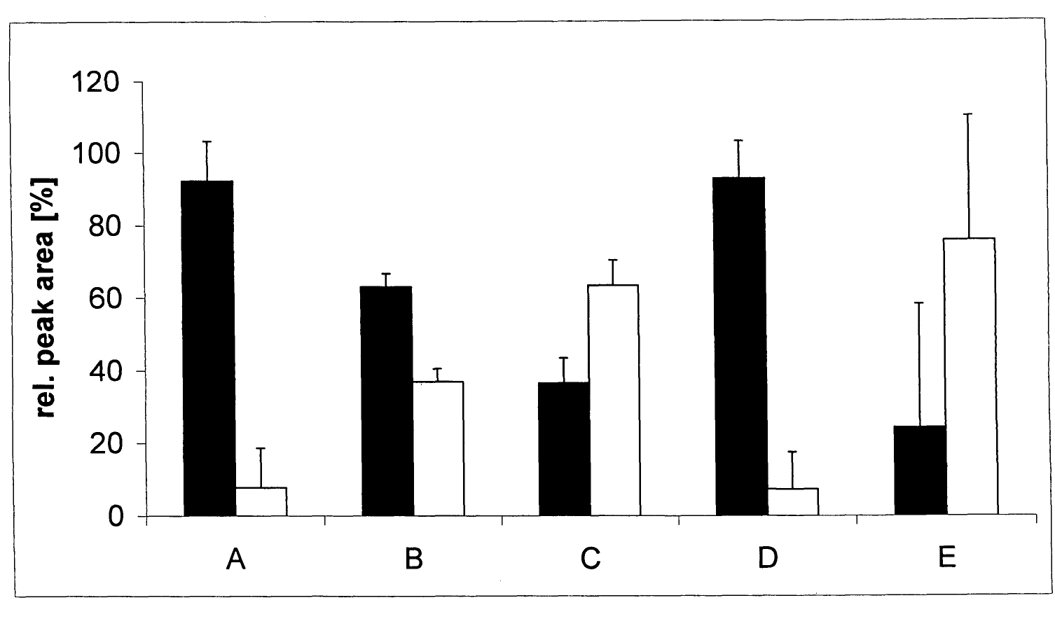

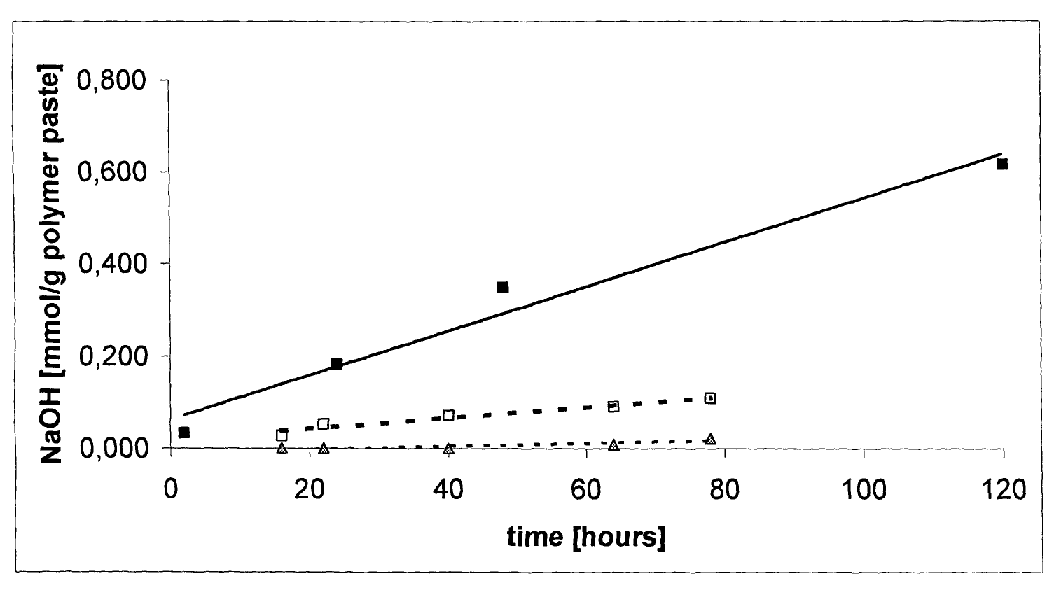

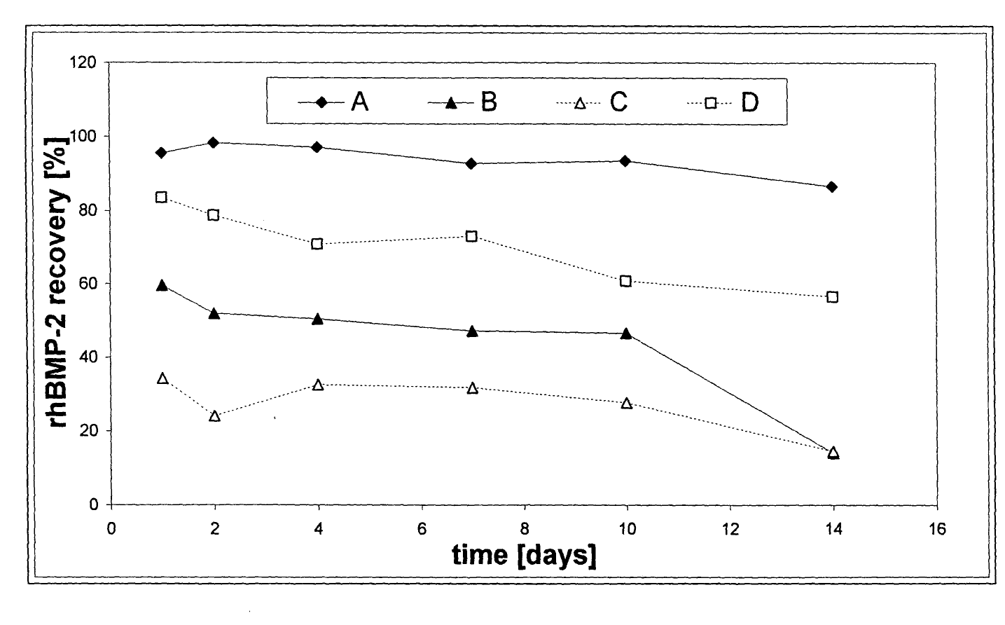

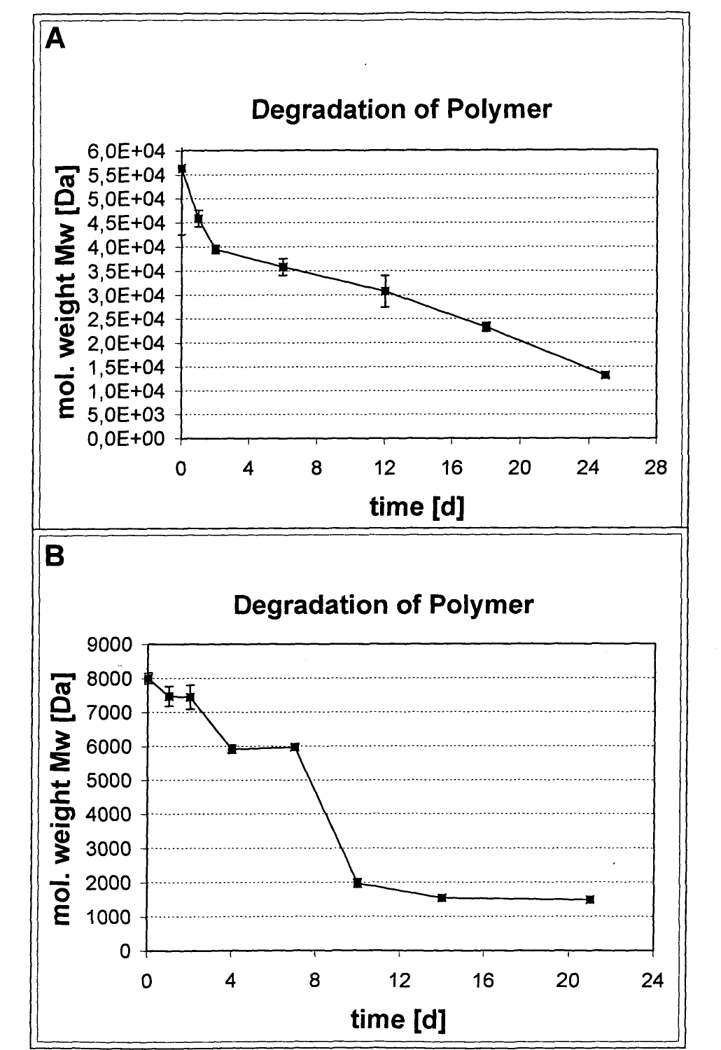

Image A shows the outer surface of the two component moldable biomaterial of the present invention, exhibiting additional pores compared to the implant material of FIG. 1, formed by the swelling of carboxymethylcellulose sodium salt. As these pores have diameters larger than 100 μm a basic requirement for the ingrowth of cells is fulfilled.

The advantage of the addition of a swelling agent such as carboxymethylcellulose sodium salt is an increase of porosity in the outer surface of the implant material whereas the inner porosity (image B) might not necessarily be increased upon addition of the swelling agent. The inner porosity is already established by the granular bed formation of the solid filler such as beta-tricalcium phosphate and by the solvent exchange out of the biodegradable paste material.

FIG. 3 shows a comparison of the mechanical stability of a polymeric paste and of the two component moldable biomaterial of the present invention 2 hours after in-situ hardening. The white column represents the polymeric paste manufactured according to example 2 with the following composition: poly(-lactic-co-glycolic-acid) with a lactic-/glycolic acid ratio of 50:50 (RG502H) and a molecular weight of 13.6 kDa (21.6 wt %), polyethylene glycol 400 (43.1 wt %), beta-tricalcium phosphate powder (32.3%) and carboxymethlycellulose sodium salt (3.0 wt %). The black column represents an implant material manufactured according to example 3 with the following composition: beta-tricalcium phosphate granules manufactured according to example 1 (40.0 wt %) and the polymeric (biodegradable) paste (60.0 wt %) described for the white column were combined according to example 3.

It is an aspect of the present invention that though the polymer content of the implant material is decreased compared to the polymer paste, the implant material surprisingly exhibits a hardness after 2 hours, which is 2.5 fold higher than the hardness of the polymer paste without the addition of the porous ceramic material.

FIG. 4 shows the protein stability depending on the organic solvent used for the manufacture of the biodegradable paste, i.e. component b) of the moldable biomaterial of the present invention. The paste shown in FIG. 4 was prepared as described under example 6. A represents the control sample, B polyethylene glycol 400, C N-methylpyrrolidone, D dimethyl sulphoxide, E tetrahydrofurfuryl alcohol polyethylene glycol ether.

The diagram underlines that the contact between an organic solvent and a protein can provoke the (partial) degradation of the latter. As the diagram reveals, the degradation rate (white columns) can reach a level of up to 75% of the initial amount of applied protein after 48 h.

One advantage of the present invention is that a negative influence of the organic solvent onto the active substance contained in the implant material can be omitted by separating the polymer paste containing the organic solvent and the active substance containing ceramic material such as beta-tricalcium phosphate granules during storage. By mixture of the active substance containing ceramic material with the organic solvent containing paste shortly before application of the implant material to the patient the activity of the active substance such as a bone growth inducing protein can be conserved compared to a combination of the protein to the organic solvent containing matrix.

FIG. 5 represents the variability of the degree of hydrolysis of the polymer in the paste component of the moldable biomaterial of the present invention. The degree of hydrolysis was determined by the amount of sodium hydroxide solution required to neutralize the acidic degradation products of 1 g of the paste component of the moldable biomaterial. In the FIG. 5 a PGLA-copolymer was used as polymer component of the biodegradable paste material (see example 7).

Whereas the grey triangles represent a paste component of the moldable biomaterial composed of a lactic-/glycolic acid ratio of 50:50 and a molecular weight of 13.6 kDa (33.3 wt %) and polyethylene glycol 400 (66.6 wt %), the white squares represent a paste component of the moldable biomaterial of the present invention composed of a poly(-lactic-co-glycolic-acid) with a lactic-/glycolic acid ratio of 50:50 and a molecular weight of 13.6 kDa (22.2 wt %), polyethylene glycol 400 (44.5 wt %), beta-tricalcium phosphate powder (33.3 wt %) and the black squares represent a paste component of the moldable biomaterial of the present invention composed of a poly(-lactic-co-glycolic-acid) with a lactic-/glycolic acid ratio of 50:50 and a molecular weight of 13.6 kDa (21.6 wt %), polyethylene glycol 400 (43.1 wt %), beta-tricalcium phosphate powder (32.3%) and carboxymethlycellulose sodium salt (3.0 wt %).

The titration curves of the three samples reveal that the addition of the water insoluble inorganic filler (here beta-tricalcium phosphate) surprisingly accelerates the degradation of the polymer (here the PLGA-copolymer).

In addition, a high concentration of the water soluble degradation regulating agent, such as about 3% carboxymethylcellulose as used here in FIG. 5, accelerates the degradation of the polymer encompassed within the implant material thus altering the release profile of the active ingredient.

One advantage of the present invention is that the paste component, i.e. the biodegradable paste material and the particulate solid porous material, such as the particulate calcium phosphate mineral form a composite matrix, which is particularly advantageous for the replacement or augmentation of bone. The matrix maintains its structural (physical) integrity for a period of at least about two to three days after implantation and maintains its porous structure of calcium phosphate granules for several weeks within the biological environment in which bone replacement is occurring. By structural (physical) integrity it is meant that the shape and size of the implanted matrix is substantially maintained. This is in contrast to compositions which, immediately or shortly after implant, collapse into an amorphous non-porous mass. It is advantageous that the matrix maintains its porosity, which is important to the bone replacement or augmentation process.

Due to the biphasic degradation, the implant material of the present invention maintains a porous structure for improved bone formation. In addition, the biphasic degradation enables a controlled release or delivery of active substances such as bone growth inducing agents to the surrounding tissue. The release due to the first phase degradation of the polymer within the paste component of the moldable biomaterial of the present invention after in-situ hardening can be varied by varying the water insoluble solid filler and/or the water soluble degradation regulating agent.

FIG. 6 shows the recovery of rhBMP-2 bound to various biomaterials.

As FIG. 6 reveals, samples containing only beta-TCP granules exhibited nearly no interactions with rhBMP-2 (E. coli), i.e. almost 100% recovery of rhBMP-2 from the supernatant (A). Since rhBMP-2 is positively charged, reduced recovery may be triggered by negatively charged groups in the moldable biomaterial of the present invention.

It is shown that non-end-capped polymers and CMC are suitable means for triggering and/or improving the active agent adsorption of rhBMP-2 to the moldable biomaterial of the present invention. Improved adsorption of the active agent is correlated with prolonging the sustained release of the active agent from the moldable biomaterial of the present invention upon use in vivo.

Since the absolute amount of beta-TCP granules was equal for each sample, and samples containing only beta-TCP granules exhibited nearly no interactions with rhBMP-2 (E. coli, the observed adsorption of the protein to the other carriers have to be triggered by the anionic carboxylic groups introduced by non-end-capped PLGA-copolymer and CMC, respectively.

In fact, the experiment shows that the observed adsorption of the protein to the other carriers (B to D) is triggered by the anionic carboxylic groups introduced by non-end-capped PLGA-copolymer and CMC, respectively. This conclusion was supported by the observation, that those preparations containing an end-capped PLGA-copolymer (D) yielded an increased recovery rate compared with preparations containing a non-end-capped PLGA-copolymer (B and C).

FIG. 7 shows the degradation of the polymer of two different biomaterials over time. A represents the degradation of the biodegradable paste material consisting of: Resomer RG504 (44.0 wt %), PEG 400 (22.0 wt %), Biocement D (20.6 wt %), dried calcium sulfate dehydrate (20.6 wt %) and carboxymethyl cellulose sodium salt (1.0 wt %) manufactured according to example 2. B shows the degradation of the moldable biomaterial of example 8.

The data show that the degradation over time of the polymer within the biomaterials is prolonged for A compared to B leading thus to an earlier resorption of the material B. The data show also exemplarily the triphasic degradation kinetics of the moldable biomaterial of the present invention (see FIG. 7B, see decrease steps at 0-1 d, 2-4 d and 7-10 d).

### EXAMPLES

### Example 1

### Manufacturing of Active Agent Coated Particulate Solid Porous Material

This examples uses beta-TCP coated granules as solid porous material and rhGDF-5 as active agent. Alternatives can be prepared in analogy.

The raw materials have to be sterilized in an appropriate way. Initially 500 mg beta-TCP (500-1000 μm granule size) were placed in a dry form in a 2R-glass. The stock solution of rhGDF-5 (3.4 mg/ml in 10 mM HCl) was diluted to 0.54 μg/ml with the means of the corresponding coating buffer. 475 μl of the rhGDF-5 solution obtained in that manner were pipetted on the beta-TCP and absorbed. The damp granulate was incubated for 1 hour at 25° C. and then lyophilized. Other examples of coating beta-TCP are described in WO 03/043673 and PCT/EP2005/006204.

### Example 2

### Manufacturing of the Biodegradable Paste Material

Initially polymer (RG502H; PLGA; polymer composition: 48-52 mol % D,L-Lactide and 48-52 mol % Glycolide; inherent viscosity: 0.16-0.24 dl/g, 25° C., 0.1% in CHCl₃; (Boehringer, Ingelheim) was added to the obligate amount of organic solvent (PEG 400) in a porcelain crucible. These two components were homogenised and were heated at a temperature of approximately 60° C. until the polymer was completely solved in the organic solvent. Subsequently the inorganic filler (beta-tricalcium phosphate powder) and optionally other excipients (e.g. degradation regulating agents like carboxymethylcellulose sodium salt) were dispersed in the polymeric solution.

### Example 3

### In-situ Hardening Moldable Biomaterial Comprising a Porous Calcium ceramic

The coated beta-tricalcium phosphate granules of example 1 and the biodegradable paste material of example 2 were homogenized in a crucible by gentle mixing using for example a sterile spatula to form a coherent and moldable material. Different implant materials with varying ratios of beta-tricalcium phosphate granules to polymer paste (wt %/wt %) were prepared: a) a ratio beta-TCP: polymer paste of 1:1.3, b) of 1:1.4, c) of 1:1.5 and d) 1:1.7.

For all experiments requiring a biodegradable paste material or a moldable biomaterial in its hardened shape, the material was transferred into wells of a 48-well plate (250-300 mg/well). The well plate was then incubated in a bath containing PBS-buffer, whereby the temperature was fixed at 37° C. The bath was constantly shaked applying a frequency of 150 min⁻¹.

### Example 4

### Mechanical Testing

The hardened and moist specimens of the biodegradable paste material, prepared as described in example 2 and the in-situ hardening biodegradable paste material (implant material) prepared as described in example 3, were transferred into wells of a 96-well plate (150-200 mg per well, three wells per time point and sample). Subsequently the well plate containing the samples was transferred into an incubation bath, which was constantly remained at 37° C. to simulate physiological conditions, whereas PBS-buffer served as an incubation media. At pre-defined times the 96-well plate was removed from the incubation bath to carry out the mechanical testing.

Hardness of the specimens was tested by using a TH 2730 (Fa Thuemler). Substantially this machine consists of a metallic punching tool, which enables to apply compressive forces on the specimens and a LVDT-transducer, which serves to control and to measure the applied force and to determine the distance, covered during the measurement. Prior to testing the different specimens, the height (h₁) of a well, which does not contain any specimen has to be defined. Therefore the starting point of the punching tool for the following measurements was fixed. The actual determination of hardness of the specimens encompasses two steps. In a first measurement the height of the particular specimen (h₂) has to be ascertained, whereas the crosshead velocity of the punching tool was 40 mm per minute and the applied force was limited to 0.2 N. A second measurement was carried out to determine the distance (d), covered by the punching tool within the specimen during a period of 30 seconds, whereby the applied force was kept constant at 20 N. Hardness of the specimen was calculated in the following manner:

hardness [%]=(h₂−d)/h₂*100%

The described method was based on the determination of hardness according to Shore (DIN 53505).

### Example 5

### Preparation for SEM-Analysis

The hardened and vacuum dried specimens were sputtered with gold according to a standard procedure known for experts in the field. The SEM-micrograms were performed applying a voltage of 20 kV. The target structures for these analyses were the surface and the core of the particular specimens of the implant material and especially the porosity exhibited by these structures.

### Example 6

### Stability of rhGDF-5 in Different Organic Solvents

Solvents such as polyethylene glycol 400, N-methylpyrrolidone, dimethyl sulphoxide and tetrahydrofurfuryl alcohol polyethylene glycol ether were used. The samples as well as the references were prepared by coating 500 mg of beta-TCP with rhGDF-5 to achieve a final concentration of 500 μg/g beta-TCP. Afterwards 666 μl of the respective solvent were added to each sample, while the references were left untreated. After incubation for 24 hours at a temperature of 25° C. both samples and references were extracted at 4° C. for one hour with 3 ml of an extraction buffer, consisting of urea (8 M), Tris (10 mM) and EDTA (100 mM), whose pH level was adjusted to 6.7 with hydrochloric acid. After this extraction step all samples and references were centrifuged for 3 minutes with 4500 rpm. Subsequently the supernatant was diluted with solvent A (0.15% trifluoroacetic acid and 20% acetonitrile in water) in a ratio of 1:1. Solvent B was composed of 0.15% trifluoroacetic acid and 84% acetonitrile in water. The characterization of the proteins was carried out using a Vydac C18, 2.1×250 mm at a flow rate of 0.3 ml/min. The elution profile was recorded by measuring the absorbance at 220 nm. The amounts of rhGDF-5, rhBMP-2 and their degradation products were calculated from the peak area at 220 nm.

### Example 7

### Determination of Polymer Degradation

The biodegradable paste material, manufactured as described in example 2, was accurately weight in a 6R-vail to which about 3.0 ml of PBS-buffer were added. To indicate the pH-value of the sample 20 pH of bromothymol blue were added to the sample, whereas a deep blue colour indicated a neutral pH. The degradation of the polymer (here: PLGA-copolymer) provokes a decrease of the pH-value, which is indicated by a colour change from deep blue to yellow. To defined time points the supernatant of the sample was titrated with 0.04 M sodium hydroxide solution until the pH value of the sample reached a neutral level indicated by a deep blue colour of the indicator. The whole consumption of sodium hydroxide was summated up to each time point and was normalized by considering the applied mass of the PLGA-copolymer.

### Example 8

### Determination of In-vitro Polymer Degradation and Quantification of the Polymer Content within the Moldable Biomaterial Over Time

10.0 g beta-TCP granules and 15.0 g biodegradable paste material (Resomer RG502H (22.2 wt %), polyethylene glycol 400 (44.5 wt %), beta-TCP powder (20.8 wt %) and dried calcium sulphate dihydrate (12.5 wt %)) were admixed. Portions of 1.0 g of the resulting coherent mass were taken to form cylindrically shaped specimens, which were subsequently transferred into a 50 ml polypropylene reaction tube filled up with 50 ml of physiological phosphate buffer.

At designated time points (after 1 d, 2 d, 4 d, 7 d, 10 d, 14 d, 21 d of incubation) specimens were rejected and vacuum dried. Approximately 75 mg of the vacuum dried composite material were accurately weighed in a 1.5 ml polypropylene reaction tube. Subsequently 1.0 ml of tetrahydrofuran were added. The samples were incubated for 10 minutes at ambient temperature under constant horizontal agitation (300 min⁻¹). The insoluble inorganic components were separated from the polymer solution by centrifugation at 13000 rpm for 5 minutes. The obtained supernatant was then subjected to analysis via a combined size exclusion chromatography multi angle light scattering facility, essentially consisting of a HPLC-device, a size exclusion column (7.8 mm*30.0 cm) and a multi angle light scattering detector serially combined with a refractive index detector.

To determine the molecular weight of the polymer extracted from the respective samples, 200 μl of the supernatant were injected. Thereby the polymer was eluted by tetrahydrofuran applying a constant flow rate of 1.0 ml/min. The column temperature added up to 40° C. To enable the employed software to calculate the absolute molecular weight and the absolute injected amount of the analysed polymer, the differential index of refraction (dn/dc) of the respective polymer was determined previously by recording the area under the curve of the refractive index signal for various polymer concentration. By proceeding analogously with the organic plasticizer this method allows the determination of the relative composition of the moldable biomaterial over time.

### Example 9

### Interaction of rhBMP-2 (E. coli) with Moldable Biomaterials of Various compositions

75 mg of beta-TCP granules were mixed with 112.5 mg of the biodegradable paste material manufactured according to the above samples to obtain the moldable biomaterial. Thereby following variations of the biomaterial were employed:
A) beta-TCP granules
B) beta-TCP granules+biodegradable paste material composed of PEG 400 (44.5 wt %), beta-TCP powder (33.3 wt %) and Resomer® RG502H (non-end-capped, 22.2 wt % purchased by Boehringer Ingelheim)
C) beta-TCP granules+biodegradable paste material composed of PEG 400 (43.0 wt %), beta-TCP powder (32.4 wt %), Resomer® RG502H (non-end-capped, 21.6 wt % purchased by Boehringer Ingelheim) and carboxymethly cellulose sodium salt (CMC) with a DS of 0.7 and a particle size of 100-200 μm (3.0 wt %)
D) beta-TCP granules+biodegradable paste material composed of PEG 400 (44.5 wt %), beta-TCP powder (33.3 wt %) and Resomer® RG502 (end-capped, 22.2 wt % purchased by Boehringer Ingelheim)

To discriminate the specific impact of the biodegradable paste material on the extent of interaction with rhBMP-2 (E. coli) from the distribution of beta-TCP granules to overall protein adsorption, 75 mg beta-TCP were applied as a reference carrier (A).

Each sample was transferred into a 15 ml polypropylene reaction tube filled with 15 ml of an aqueous buffer (60 mM calcium chloride in 20 mM morpholinoethanesulfonic acid monohydrate (MES) solution, 0.01 wt % polysorbate 80, 0.02 wt % sodium azide, pH 6.2). All samples were spiked with 30 μg rhBMP-2 (E. coli). At designated time points (1 d, 2 d, 4 d, 7 d, 10 d) the rhBMP-2 (E. coli) concentration in the supernatant of each sample was determined by RP-HPLC using a 250 mm*4.6 mm C4 column (Vydac).

20 wt % acetonitrile and 0.15 wt % trifluoroacetic acid in water and 84 wt % acetonitrile and 0.15 wt % trifluoroacetic acid in water respectively served as eluents. The flow added up to 0.8 ml/min. The concentration was determined by means of fluorescence detection at 340 nm (excitation 280 nm). The amount of rhBMP-2 in the supernatant was determined in relation to the amount of rhBMP-2 in the supernatant at time point “zero” (100% recovery).

1. A moldable biomaterial comprising
a) a particulate solid porous material with a particle size of 100-4000 μm and
b) a biodegradable paste material.

2. The moldable biomaterial of claim 1, wherein
a) the particulate solid porous material comprises ceramic granules, made of tricalcium phosphate with an average particle size of 100-4000 μm; and
b) the biodegradable paste material is a paste comprising
ii. a plasticizer, which is a water soluble or water miscible biocompatible organic liquid;
iii. a water insoluble polymer, which is soluble in the plasticizer and which is biocompatible, biodegradable, and/or bioresorbable; and
iiii. a water insoluble solid filler, which is insoluble in the plasticizer.

3. The moldable biomaterial of claim 1, which has a moldable consistency, and which is capable of hardening in-situ to form a solid implant upon contact with an aqueous medium or a body fluid.

4. The moldable biomaterial of claim 1, wherein the components a) and b) are used in a ratio in order to form a coherent product.

5. The moldable biomaterial of claim 1, wherein the paste of component b) comprises a water soluble degradation regulating agent, which is carboxymethylcellulose.

6. The moldable biomaterial of claim 1, further comprising c) an active agent.

7. The moldable biomaterial of claim 6, wherein said active agent is a bone growth factor.

8. The moldable biomaterial of claim 6, wherein said active agent selected from the group consisting of BMP2, BMP7 and GDF5.

9. The moldable biomaterial of claim 1, which shows biphasic degradation in-situ.

10. The moldable biomaterial of claim 1, which maintains a physical integrity for a period of at least 2 to 3 days after hardening in-situ and which maintains a porous granular structure after degradation of the polymeric component.

11. A kit comprising the isolated components a) and b) of the moldable biomaterial as set forth in claim 1.

12. An implant comprising the components a) and b) of the moldable biomaterial as set forth in claim 1.

13. A method of manufacturing a moldable biomaterial comprising mixing a paste comprising
i. a plasticizer, which is a water soluble or water miscible biocompatible organic liquid;
ii. a water insoluble polymer, which is soluble in the plasticizer and which is biocompatible, biodegradable, and/or bioresorbable; and
iii. a water insoluble solid filler, which is insoluble in the plasticizer,
with calcium phosphate or calcium sulfate
so that the mixture has a moldable consistency, which is capable of hardening in-situ to form a solid porous implant upon contact with the aqueous medium or body fluid.

14. The method of claim 13, wherein the paste is water dried and/or manufactured using water free components (i), (ii) and/or (iii).

15. Use of the moldable biomaterial of claim 1 for the manufacture of a pharmaceutical composition or a medical device to be used for spinal fusion, long bone defects, critical size defects, non-union, joint relocation preferably knee or hip relocation, fracture repair, cartilage repair, maxillofacial reconstruction, periodontal repair, degenerative disc disease, spondylolisthesis, bone void filling.

16. A moldable biomaterial manufactured by the method of claim 13.

17. A kit comprising the isolated components of a), b) and c) of the moldable biomaterial as set forth in claim 6.

17. An implant comprising the components of a), b) and c) of the moldable biomaterial as set forth in claim 6.
